(19) United States
(12) Patent Application Publication
(10) Pub. No.: US 2024/0252140 A1
(43) Pub. Date: Aug. 1, 2024

(54) DEVICE AND METHOD FOR THE DIAGNOSIS OF A PNEUMONIA BY ANALYSIS OF ULTRASOUND IMAGES

(71) Applicant: Imedicals S.r.l., Lecce (IT)

(72) Inventor: Sergio Casciaro, Lecce (IT)

(73) Assignee: Imedicals S.r.l., Lecce (IT)

(21) Appl. No.: 18/289,428

(22) PCT Filed: May 4, 2021
(86) PCT No.: PCT/IB2021/053701

§ 371 Date: Nov. 3, 2023

(51) Int. Cl. A61B 8/08; A61B 8/00; G01S 7/52; G01S 15/89; G06T 7/00; G06T 7/11

(52) U.S. Cl. CPC A61B 8/08; A61B 8/4488; A61B 8/5207; A61B 8/5223; A61B 8/5261; G01S 7/52036; G01S 15/8915; G06T 7/0012; G06T 7/11; G06T 2207/10132; G06T 2207/30061

(57) ABSTRACT

A method for calculating a diagnostic parameter for pneumonia, comprising the steps of: (100) acquiring a plurality of ultrasound images of the lung in a first acquisition position, (200) individuating inside each image the area under the pleural line; (300) individuating a plurality of ultrasound makers (C1, . . . , Cn) therein; (400) calculating for each marker (C1, . . . , Cn) a plurality of features (C11, . . . , C1m, . . . , Cn1, . . . , Cnm) (450) calculating at least another parameter relating to all the images acquired at point (100) as a whole, comprising the percentage of pleural line interested by “White lung”; (500) calculating a diagnostic parameter with a two steps procedure: a first step, in which to each image a first value of Pneumonia Score is assigned and a second step in which the average of the first values calculated for each image acquired at point (100) is corrected as a function of said further parameter calculated at point (450).

### TECHNICAL FIELD

The present invention relates to an ultrasound image and/or relative “raw” ultrasonic signal analysis, configured to allow a numeric diagnostic parameter automatized calculation, indicating the possible presence and the progression stage of COVID-19 pneumonia, i.e. caused by SARS-CoV-2 virus, while distinguishing other types of pneumonia, which can be individuated specifically and by means of the same method as well.

### STATE OF THE ART

Thanks to their high sensibility, chest X-ray and computed tomography (CT) are currently the imaging techniques of choice to diagnose and monitor both common and COVID-19 pneumonia (Coronavirus Disease caused by SARS-CoV-2).

However, both chest X-ray and CT have some important limitations due to limited accessibility, high ionizing radiation doses and high costs, which do not allow their usage for screening purposes. Moreover, it could be difficult for patients suffering from hypoxemia and/or heart failure to undergo a CT, and it is also difficult to transfer patients from intensive care unit to CT system and, anyway, due to the connected infection risks, important transfer limitations remain.

Lung ultrasound has shown a promising capacity of diagnosing and monitoring COVID-19 patients, similar to chest CT and superior to standard chest radiography used to evaluate pneumonia and/or adult respiratory distress syndrome. Lung ultrasound can overcome the main CT limitations, since it can be brought to patient's home or bed, it is accurate, radiation-free, cheaper, and so it could become the imaging method of choice for patients, whose triage has to be carried out at home or in critical conditions in intensive care units, or however for old patients and for specific categories, such as pregnant women and children.

It is known (Peng et al., “Findings of lung ultrasonography of novel coronavirus pneumonia during the 2019-2020 epidemic”—Intensive Care Med 2020) that COVID-19 pneumonia, on the ultrasound images obtained by scanning various points of the rib cage, with specific diffusion “patterns” produces:
a pleural line thickening, with irregular margins,
pleural effusions,
a plurality of vertical lines, called B-lines (or also “lung comets”), which can be focal, multi-focal or confluent;
consolidation areas (also called “consolidative areas” or “consolidations”) in a plurality of different configurations (patterns) including small multi-focal consolidation areas and wider not trans-lobular ones (also called “intra-lobular”), trans-lobular (also called “inter-lobular”) consolidation areas with possible dynamic air bronchograms.

A lung consolidation is observed when the lung ventilation degree is reduced, and air is substituted by exudate/inflammatory cells in the alveoli. Thus, the ultrasound beam is able to penetrate the parenchyma highlighting a “consolidation” with ultrasound pattern structure similar to the liver one (pulmonary hepatization). In the B-mode ultrasound, the pulmonary parenchymal consolidation is associated to the following phenomena:
pleural line interruption/interruptions;
presence of one or more homogeneous hypoechoic areas (similar to liver tissue) under the pleural line interruptions;
punctiform and/or linear hyperechoic areas (called “air bronchograms”) on the deepest edge of the hypoechoic area and/or inside the same;
elongated white areas with “cascade” effect, which go from hyperechoic areas to the bottom of the B-Mode image.
The above described effects are amplified as a function of the disease severity, as it is schematized for simplicity and as a way of example in the following table, which describes in detail the aspect of the different stages of pulmonary consolidation on the ultrasound images:

| | | Hypoechoic | Hyperechoic | Cascade |
|---|---|---|---|---|
| stage | Pleural line | area | areas | effect |
| 1 | One or two | Non-essential | Little areas | Visible with |
| | interruptions of | or little | present, very | moderate |
| | small dimensions, | extended | close to pleural | intensity, |
| | visible in the | area | line | starting very |
| | B-mode field | | | close to |
| | of view | | | pleural line |
| 2 | At least two | One or more | One or more | Increase of |
| | visible | areas with | areas clearly | intensity and |
| | interruptions | greater | not adjacent to | of extension |
| | of pleural line. | extension | pleural line | of the effect |
| | Dimensional | | | |
| | increase of the | | | |
| | interruptions | | | |
| 3 | Unification and | Very | Many punctiform, | High |
| | increase of | extended | spot and/or | intensity. |
| | interruptions; | area | linear areas, | Start very far |
| | increase of | | both inside the | from pleural |
| | pleural line | | hypoechoic area | line |
| | interrupted | | and on the | |
| | portion | | deepest edge | |

Moreover, during the recovering stage from COVID-19 pneumonia, on the ultrasound images it is also possible to observe:
“A-lines” occurrence (substantially horizontal lines, or anyway perpendicular to the propagation direction of the ultrasonic beam emitted by the probe, and substantially parallel to the pleural line).

In FIG. 1, some lung ultrasound images of a patient with confirmed COVID-19 pneumonia are shown.

In the two upper images, typical wide vertical artifacts can be observed, which start from pleural line or small sub-pleural consolidations. The vertical artifacts origin is not punctual. In the lower images, it is observed how the pleural line is interrupted by more visible consolidations. It is observed that from these consolidations wide vertical artifacts start, overlapping with a white lung area. Further ultrasound images relative to a patient with confirmed Coronavirus pneumonia are shown in FIG. 2.

Cisneros-Velarde et al., IEEE-EMBC 2016, describe a video analysis method that identifies two markers (pleural line and lung consolidations), calculates an adaptation parable for pleural line and the center of gravity position of all the consolidations detected, and classifies each video as “healthy”, “ill” or “possibly ill” as a function of the statistics associated to the adapted parable and to the center of gravity position.

Malena Correa et al., PLOS ONE 2018, describe a method for automatic classification of pediatric pneumonia, based on the analysis of brightness distribution schemes in rectangular image segments. US2020/054306 describes an interpretation method of ultrasound scan videos by means of one or more convolutional neural networks for detecting A-lines, B-lines, pleural line, consolidations and pleural effusions for obtaining a lung and diagnosis by using a neural network.

WANG et al., Journal of Cardiothoracic and Vascular Anesthesia 2020, describe some characteristics of lung ultrasound in patients suffering from COVID-19. They state that lung ultrasound characteristics in patients with COVID-19 vary significantly from patient to patient and from day to day, and that the ultrasound images of lung lesions in patients with COVID-19 are: pleural line thickening and irregularity; presence of B-lines and consolidations in various forms; occurrence of A-lines during recovering stage.

### Technical Problem

Anyway, the usage of lung ultrasound has various limitations as well, as explained for example in Bouhemad et al., “Clinical review: Bedside lung ultrasound in critical care practice”, Crit Care. 2007; 11(1):205.

In primis, an irregular B-line or a consolidation pattern can be observed in any pneumonia or interstitial lung disease, even not associated to COVID-19, and it is nearly impossible, even for a skilled healthcare operator, to distinguish between the various disease types only on the basis of the subjective image analysis.

The lung ultrasound requires also an intensive training in simple applications of at least six weeks of the healthcare operator, in order to allow him to acquire the needed knowledge and skills; moreover, currently, even if some ultrasound markers, occurring in the ultrasound images of patients with COVID-19, have been identified, there are no quantitative indicators available of the disease severity deriving from the images, and so the diagnosis remains of qualitative type and its reliability strongly depends on the healthcare operator expertise.

### Aim of the Invention

Therefore, object of the present invention is a method for the analysis of ultrasound images and/or relative unfiltered ultrasonic signals (so called “raw” or “radiofrequency” ultrasonic signals), which allows to obtain a quantitative evaluation of lung tissues condition. The present invention provides also an ultrasound device comprising computing means on which computer programs, configured to carry out such method, are loaded. Yet, the invention provides a method for the analysis of lung ultrasound images and/or relative unfiltered ultrasonic signals, configured to calculate at least one quantitative diagnostic parameter, indicating the possible presence of a lung disease, whether it is caused by SARS-CoV-2 virus or by any other cause, and its clinical stage.

According to another aim, the present invention provides a method for the analysis of lung ultrasound images that presents all the just described advantages and whose results are highly repeatable and independent of the healthcare operator expertise.

Another advantage is that the method according to the invention does not require, for its own implementation, skilled ultrasound operators, since the method provides quantitative diagnostic indicators that are calculated in a fully automatic manner and are completely independent of the operator.

Another advantage is that the ultrasound acquisition for implementing the method follows a very easy protocol, during whose execution the operator is guided by the software and in which the acquisitions that have been carried out without satisfying all the protocol criteria are automatically rejected, and the operator is asked to repeat them.

In particular, the diagnostic indicators calculated with the method according to the invention allow to characterize the pneumonia, by defining if it is caused by COVID-19 or by any other type of virus or causes (for example, bacteria, parasites, other fungi, chronic obstructive pulmonary disease (COPD), etc.).

Another advantage is that the quantitative diagnostic indicators calculated by means of the method according the to invention allow an objective disease severity staging and the early identification of COVID-19 possible presence before the onset of pulmonary fibrosis in asymptomatic patients.

Finally, one of the main advantages of the method according to the invention is to allow to monitor the disease progression in a determined patient. In fact, the quantitative diagnostic indicators calculated by means of the method according to the invention include not only a descriptive indication of the disease “staging” (mild, moderate, severe, etc.) by means of a parameter called “Pneumonia Score”, associated to the lung disease severity relating to the lung tissue portion object of the scan in each acquisition position, but also a specific numeric value associated to the whole lung examination, indicating the probability that the pneumonia is caused by Covid-19 (Covid Index).

For example, assuming that Pneumonia Score is indicated as a numeric parameter in a scale from 1 to 100, having the disease in the “moderate” stage with Pneumonia Score=55 is different from having it in the same “moderate” stage but with Pneumonia score=50. If the two values were obtained on the same patient, depending on which one was obtained at first, it would be possible to understand if the disease is in progression or in remission and also, as a function of the time interval between the two acquisitions, at what speed.

As an alternative, Pneumonia Score can be indicated in a scale from 0 to 4, by classifying each lung tissue portion as healthy, with the disease in the initial, intermediate, advanced or critical stage. It has to be specified that the parameter can be referred to a specific portion of lung tissue, if calculated on the basis of acquisitions in a unique position, or to the patient whole clinical condition, if calculated on the basis of ultrasound acquisitions carried out in a plurality of positions.

The possibility to repeat the acquisition every day (and basically also more times a day), combined with the availability of said quantitative diagnostic indicator, allows to carry out a “short term” monitoring and a rapid identification of the disease progression trend, which is unthinkable for any other technology.

Moreover, a short term monitoring of the just described type is extremely useful to choose the correct patient management, also considering the limited beds in intensive care units, and to evaluate as well the actual effectiveness of a drug, also in comparison with patients treated with different approaches (due to the usage of a different drug or because of different administration doses/times of the same drug), and this would have a fundamental importance in the clinical studies aiming at the introduction of new drugs.

### DESCRIPTION OF FIGURES

FIGS. 1 and 2 show ultrasound images, obtained by employing convex probe and intercostal (transversal) acquisition on patients with COVID-19 pneumonia, and indication of consolidations and B-lines position; FIG. 3 shows a lung ultrasound image on which the pleural line identification is indicated; FIGS. 4 and 5 show two ultrasound images with indication of A-lines and B-lines, respectively; FIG. 6 shows a lung ultrasound image with longitudinal probe positioning (orthogonal to ribs); FIGS. 7 to 9 show flowcharts illustrating the steps needed to carry out the method according to the present invention; FIGS. 10, 11 and 12 show three examples of ultrasound images of the COVID-19 disease progression and relating in particular to three progressive advancement stages of lung consolidation.

### TERMS AND DEFINITIONS

It is to be said at first that in the context of the present Patent application, for “raw ultrasonic signal” or “radiofrequency ultrasonic signal” is intended an ultrasonic signal emitted by the probe and reflected by the human body towards the same probe, before the same is processed in order to obtain the ultrasound image; yet, it has to be specified that, where not stated otherwise, for “ultrasound image” it is intended an ultrasound image of B-mode type, obtained along the propagation plane of the ultrasonic beam emitted by the probe.

Yet, it has to be specified that for “raw ultrasonic signal corresponding to a determined Region of Interest (ROI)” it is intended the portion of the raw ultrasonic signal that, suitably treated, has given origin to a corresponding segment of the Region of Interest identified on the B-mode ultrasound image.

As it is known, in fact, the correlation between the position of each pixel in the ultrasound image and the ultrasonic signal that generated it, is a function of the time interval occurring between the ultrasonic impulse emission and the relative echo reception (reflected signal), since the signal reflected by tissues positioned at greater depths needs more time to reach the probe after being reflected.

Therefore, regardless of the nature of possible subsequent processing, the segmentation of the “raw ultrasonic signal corresponding to a determined ROI” occurs in the time domain, so that the portion of the raw ultrasonic signal that, in the ultrasound image, has given origin to a determined segment of the same image is isolated.

Yet, according to what commonly known at the state of the art, the used ultrasonic probe comprises an array of CMUT or piezoelectric type transducers arranged side by side, configured to emit a plurality of ultrasonic signals, so that a “line of sight” of the ultrasound image (directed up-downwards) corresponds to each signal, and the group of lines of sight, arranged side by side, allows to reassemble the ultrasound image.

It has to be specified that the processing carried out on the radiofrequency ultrasonic signals is described in the following, for shortness and description clarity's sake, with reference to a single ultrasonic signal. Obviously, also where not specifically stated, it is clear that the processing as a whole can be conveniently applied to a plurality of raw ultrasonic signals, each one received by one of the CMUT or piezoelectric transducers, included in the ultrasound probe.

It has to be precised that for “point” of the raw signal (radiofrequency ultrasonic signal), it is intended the value assumed by the raw signal in a single sampling: in a sampling at 40 MHz, as a way of example, 40,000 points are obtained for each millisecond of acquired signal.

Moreover, it has to be specified that the whole processing described in the following is carried out by means of an ultrasound system provided with at least one ultrasound probe—which can be both of convex type or linear type or also of trans-esophageal or matrix phased array type—and with suitable guiding means of said probe, with computing means for processing the signal, configured to generate the signal to be sent by means of said probe, and to analyze the signal received by said probe in order to obtain an ultrasound image, with user interaction means comprising a graphical interface and control means, as for example keyboards and/or pointing means.

These are, also as a whole, the devices known at the state of the art and commonly used in the ultrasound technique.

Anyway, it has to be precised that, in order to implement the diagnostic methods requiring a radiofrequency ultrasonic signal usage, the ultrasound device according to the invention is configured not only to process the raw ultrasonic signal (radiofrequency ultrasonic signal) to obtain an ultrasound image but also to store the raw ultrasonic signal in order to carry out following processing of the same.

Yet, it is useful to precise that in the present description, the definition “Ultrasound Dataset” refers to all the radiofrequency ultrasonic signals relating to a plurality of sequentially acquired frames of a specific patient.

From the ultrasound signals relating to each frame, according to the processing commonly known at the state of the art, the corresponding ultrasound image can be reassembled. Anyway, the raw ultrasonic signal contains further information which goes normally lost during the processing needed to obtain the ultrasound image, and so not present in the image, but which can be conveniently used to improve the efficacy of the diagnostic method according to the present invention, as explained in detail in the following.

The definition “spectrum associated to a segment of ultrasound image” refers to the frequency spectrum obtained by the transformation of the raw ultrasonic signal corresponding to a respective segment of the ultrasound image.

### Method for Calculating the Diagnostic Parameter

In the following, with reference to some preferred embodiments, the diagnostic method according to the invention is described, which can be implemented by means of a device of the just described type.

In a first embodiment (cfr. scheme in FIG. 6), the method according to the invention comprises the steps of:
(100) acquiring a plurality of ultrasound images of the lung of a patient in a first acquisition position, in which at least the pleural line and a portion of the lung below are visible.

Those images are preferably acquired according to a technique commonly known Imaging, by means of an ultrasound system provided with an ultrasound probe comprising an array of CMUT or piezoelectric transducers, each transducer being configured to emit an ultrasonic impulse directed towards the tissues object of classification and to receive the raw ultrasonic signal reflected by the tissues of the patient in response to said ultrasonic impulse; preferably, moreover, both the radiofrequency ultrasonic signal received by said transducers and said at least one ultrasound image are saved. Preferably, moreover, after step (100), the method comprises the step of
(110) considering each acquired image as acceptable for the following processing as a function of an automatized control, implemented by means of suitable programs loaded on computing means associated to said ultrasound device and configured to carry out the following operations:
(i) individuating the pleural line (whose ultrasound track is present both in healthy patients and in those with lung diseases) in the first 4 centimeters of the image depth, and carrying on with the following controls only in case of effective individuation of the pleural line, thus rejecting the image otherwise.

Even if other implementations are possible, it has to be specified that the pleural line can be individuated by:
(a) applying a “Sobel” type filter to improve the visibility of the horizontal structures;
(b) applying an “Otsu” type threshold, in order to highlight the clearer structures;
(c) carrying out two cycles of:
(i) horizontal smoothing of the image, in order to highlight further the horizontal structures as the pleura;
(ii) linear erosion, in order to remove the corpuscular and not-elongated appearing structures;
(d) identifying, starting from the bottom, the residual horizontal (continuous or patchy) structure with higher intensity;
(e) interpolating the recognized structure with a second-degree polynomial.

After individuating the pleural line, the next steps are:
(ii) verifying that the pleural line length is greater than a percentage threshold of the B-mode image width (preferably between 60% and 70%), thus rejecting the image otherwise;
(iii) verifying that the pleural line thickness is lower than a predetermined threshold (for example 5 mm), thus rejecting the image otherwise;
(iv) verifying that the pleural line average intensity is greater than a determined threshold (as a way of example, a grey average intensity>200, for 8-bit images), thus rejecting the image otherwise;
(v) in case the pleural line is detected and the previous controls are satisfied, generating normalized histograms (i.e. with total area=1) of the shades of grey relating to the tissues above and below the pleural line, and carrying out the following controls (example numeric thresholds are indicated, which can be applied in case of images acquired with “transversal” probe positioning, i.e. the probe is parallel to ribs and positioned in the intercostal space; in case of “longitudinal” positioning, i.e. with probe perpendicular to the ribs, it is possible to carry out controls with the same approach but with different numeric threshold values):
a. verifying that the portion of the histogram relating to the tissues above the pleura, representing the darkest shades of grey (for example, grey with intensity 0 to 25, on an 8-bit scale) is greater than a determined fraction of the whole area (for example 0.10), thus rejecting the image otherwise;
b. verifying that the portion of the histogram relating to the tissues above the pleura, representing the lightest shades of grey (for example, grey with intensity 230 to 255, on an 8-bit scale), is greater than a determined fraction of the whole area (for example 0.10), thus rejecting the image otherwise;
c. verifying that the portion of the histogram relating to the tissues below the pleura, representing the intermediate shades of grey (for example, grey with intensity 50 to 200, on an 8-bit scale), is greater than a determined fraction of the whole area (for example 0.9), thus rejecting the image otherwise.

Thus, the following processing described in the following is carried out only on images not rejected after said controls.

Preferably, the device according to the invention comprises also a graphical interface and is configured to communicate to the operator, by means of said graphical interface, if the acquired image has been validated or not (i.e. if the acquisition satisfies the protocol requirements and if the relative ultrasound dataset is then suitable to be analyzed to provide a diagnostic result or not). Therefore, the method comprises the steps of:
(200) individuating, in each image acquired at point (100) a Region of Interest (ROI) containing a significant lung tissue portion.

Preferably, said ROI comprises the whole area under the pleura, also because the pleura is a visible structure both in healthy and ill patients. It has to be specified that said significant portion comprises a portion of lung parenchyma, the region of lungs around the bronchus, formed by all the pulmonary lobules.
(300) Segmenting each Region of Interest individuated at point (200) in order to individuate a set of ultrasound markers (C1, . . . , Cn) therein. Conveniently, the segmentation can be carried out by means of automatic image segmentation routines. The logic used to carry out this segmentation is explained in the following, for each ultrasound marker. The computing implementation of these logics, once the same are stated, can be realized with various tools, among the ones known at the state of the art.
(400) Calculating for each one of said ultrasound markers a plurality of features (C₁₁, . . . , C₁ₘ, . . . , Cₙ₁, . . . , Cₙₘ), wherein the first subscript identifies the ultrasound marker and the second subscript identifies the specific feature relating to said characteristic.

It is convenient to precise that for “feature” it is intended a descriptive parameter which can be expressed by a numeric value relating to an ultrasound marker individuated on the ultrasound image.

During the studies needed for the development of the present invention, the analysis of a plurality of ultrasound images relating to patients with COVID-19 pneumonia at various advancement stages allowed to identify some specific features, which are particularly relevant, as a whole, to provide quantitative information useful to calculate a diagnostic parameter representing the disease stage. In the following, it is described the ultrasound marker set and respective features, relevant for calculating a diagnostic parameter according to the present invention. In the following, it will be described the preferred logic allowing these features to be used for calculating a diagnostic parameter.

C1: pleural line (whose identification on a lung ultrasound image is shown in FIG. 3).

In order to obtain an automatic segmentation algorithm, the pleural line can be conveniently identified as the horizontal interface with greater contrast and/or absolute luminosity present on the ultrasound image. A possible automatic method for pleural line individuation was explained previously.

The features associated to pleural line comprise one or more of the following ones: C₁₁: pleural line average depth; C₁₂: pleural line minimum depth; C₁₃: pleural line maximum depth; C₁₄: number of pleural line interruptions; C₁₅: pleural line absolute intensity; C₁₆: pleural line relative intensity with respect to background; C₁₇: pleural line average thickness; C₁₈: pleural line maximum thickness; C₁₉: pleural line minimum thickness; C₁₁₀: average grey value above pleural line; C₁₁₁: average grey value below pleural line; C₁₁₂: ratio between average grey values above and below pleural line; C₁₁₃: pleural line length.

C2: A-lines (whose identification on a lung ultrasound image is shown in FIG. 4). Where present, A-lines can be identified by means of an automatic segmentation algorithm which, once the pleural line is detected, analyzes the image from the pleural line downwards and by using horizontal gradient filters and contrast masks. The features associated to A-lines comprise one or more of the following ones: C₂₁: number of A-lines; C₂₂: A-lines maximum length; C₂₃: A-lines average length; C₂₄: A-lines minimum length; C₂₅: maximum vertical distance between A-lines; C₂₆: minimum vertical distance between A-lines; C₂₇: average vertical distance between A-lines; C₂₈: depth of the deepest A-line; C₂₉: distance between the most superficial A-line and the deepest one; C₂₁₀: A-line absolute intensity; C₂₁₁: A-line relative intensity with respect to background.

C3: B-lines (whose identification on a lung ultrasound image is shown in FIG. 5). Where present, B-lines can be identified by means of an automatic segmentation algorithm which, once the pleural line is detected, carries out the analysis downwards and by using vertical gradient filters and contrast masks. The features associated to B-lines comprise one or more of the following ones: C₃₁: number of B-lines; C₃₂: B-lines maximum length; C₃₃: B-lines average length; C₃₄: B-lines minimum length; C35: B-lines maximum horizontal thickness; C₃₆: B-lines minimum horizontal thickness; C₃₇: B-lines average horizontal thickness; C₃₈: number of B-lines starting from pleural line; C₃₉: ratio between the number of B-lines starting from pleural line and the number of B-lines; C₃₁₀: average contrast with respect to background; C₃₁₁: maximum contrast with respect to background; C₃₁₂: minimum contrast with respect to background; C₃₁₃: B-line absolute intensity; C₃₁₄: B-line relative intensity with respect to background.

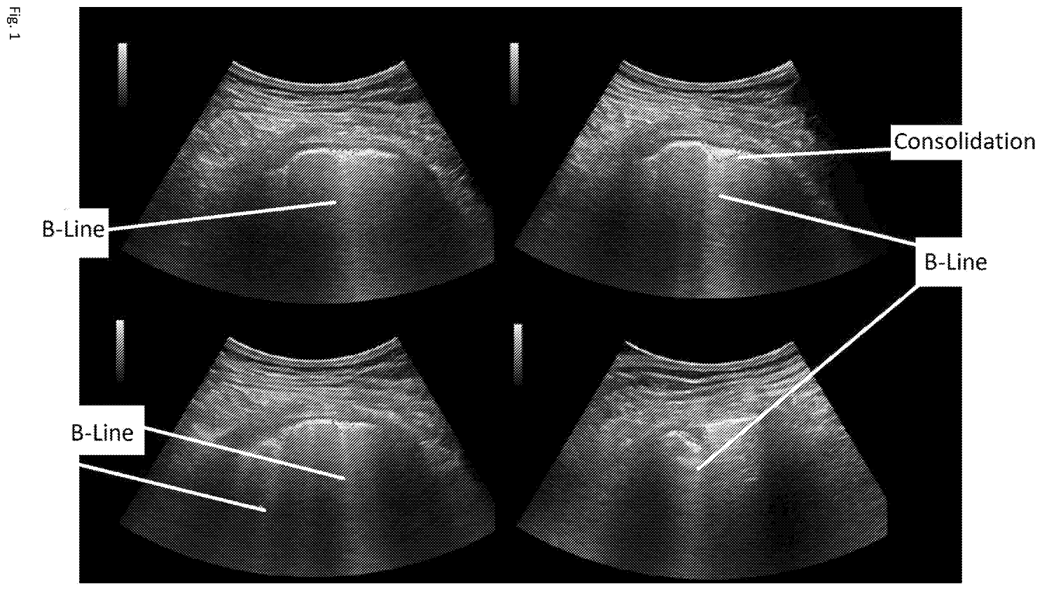

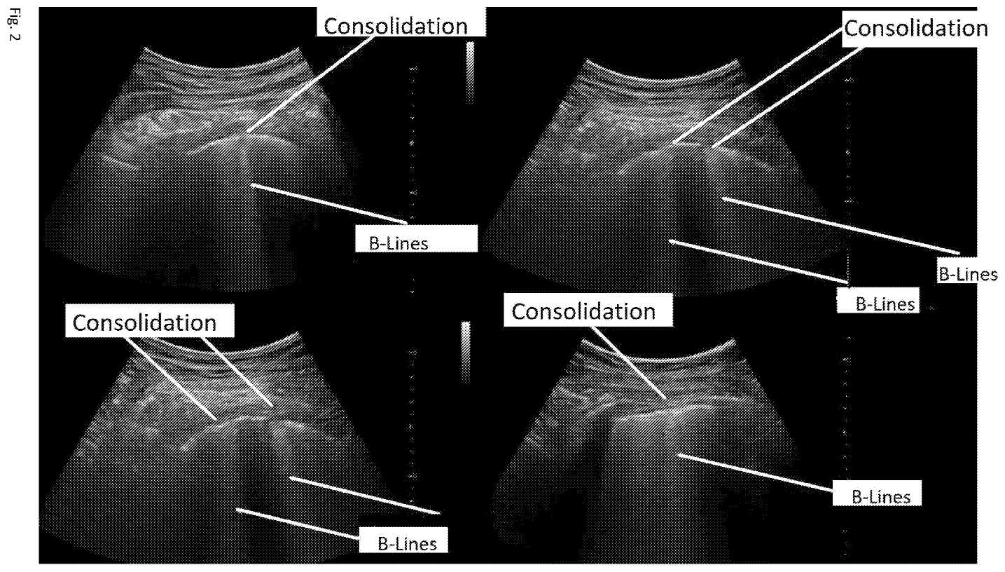

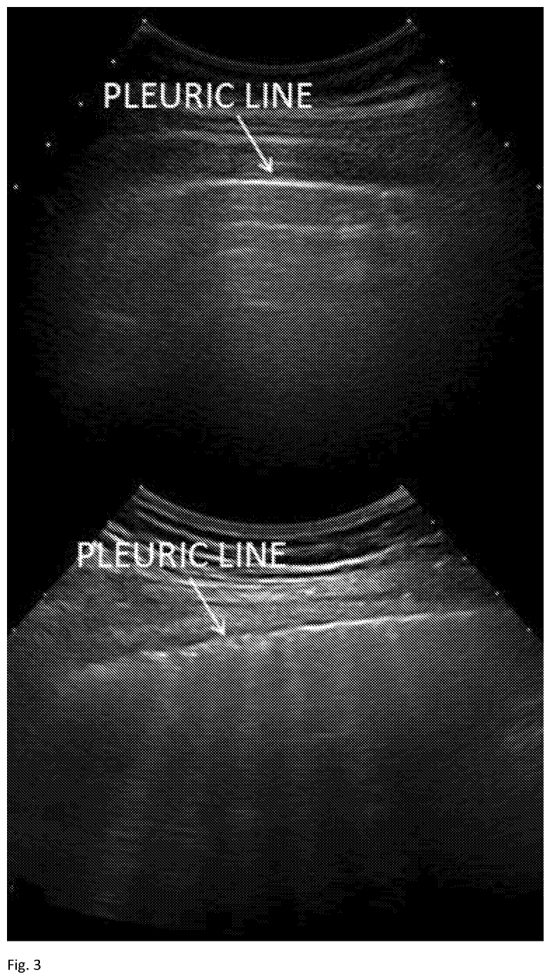

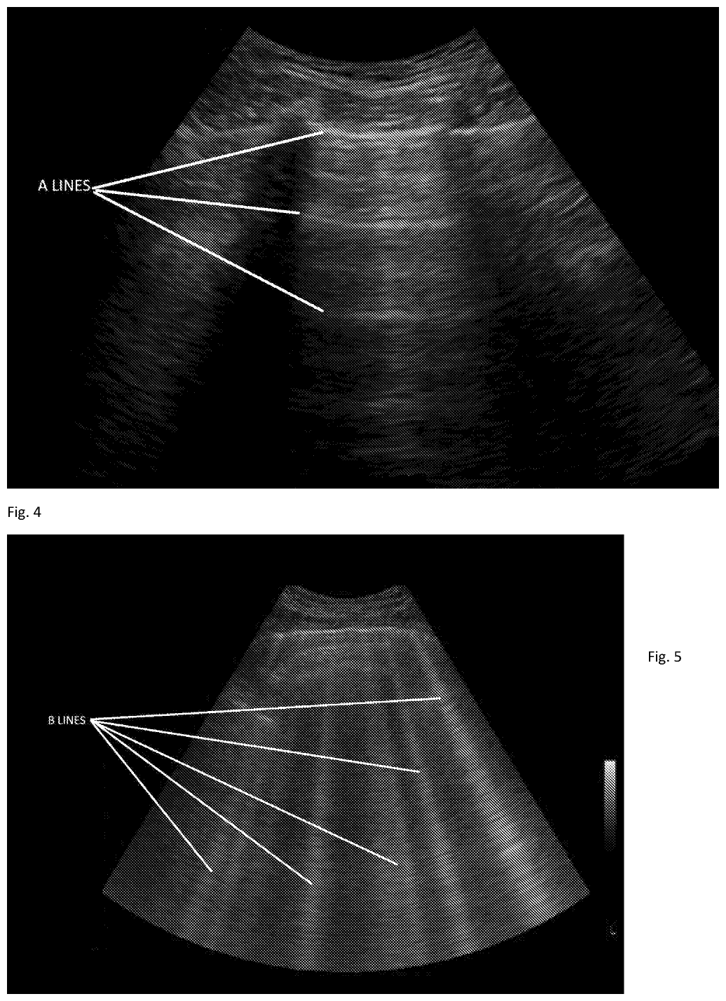

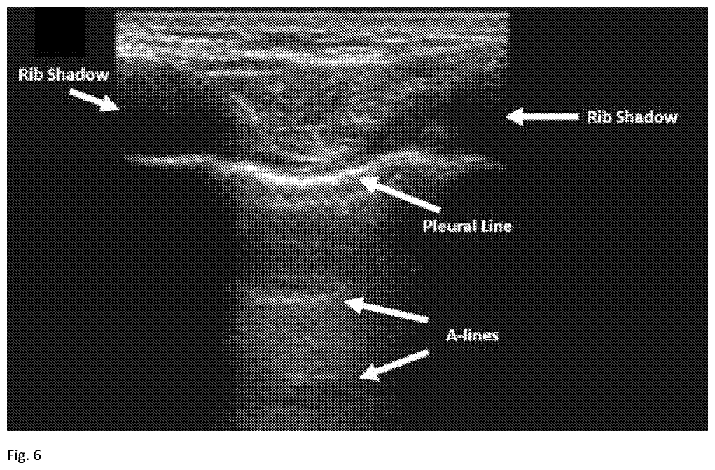

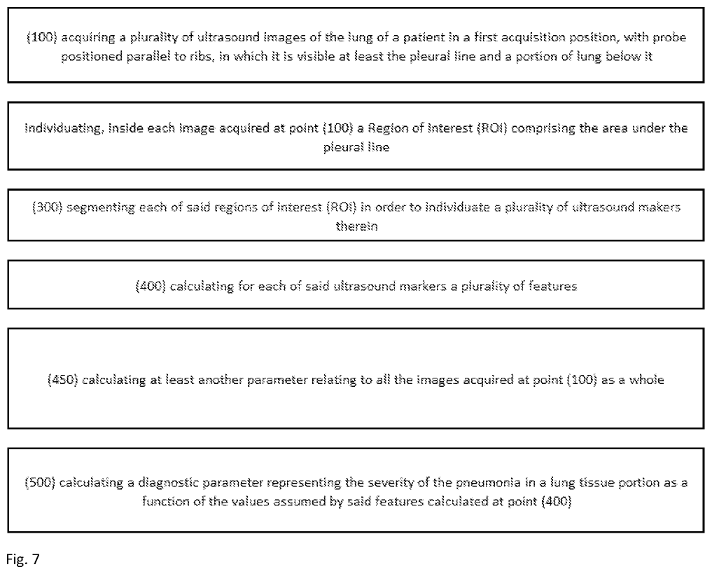

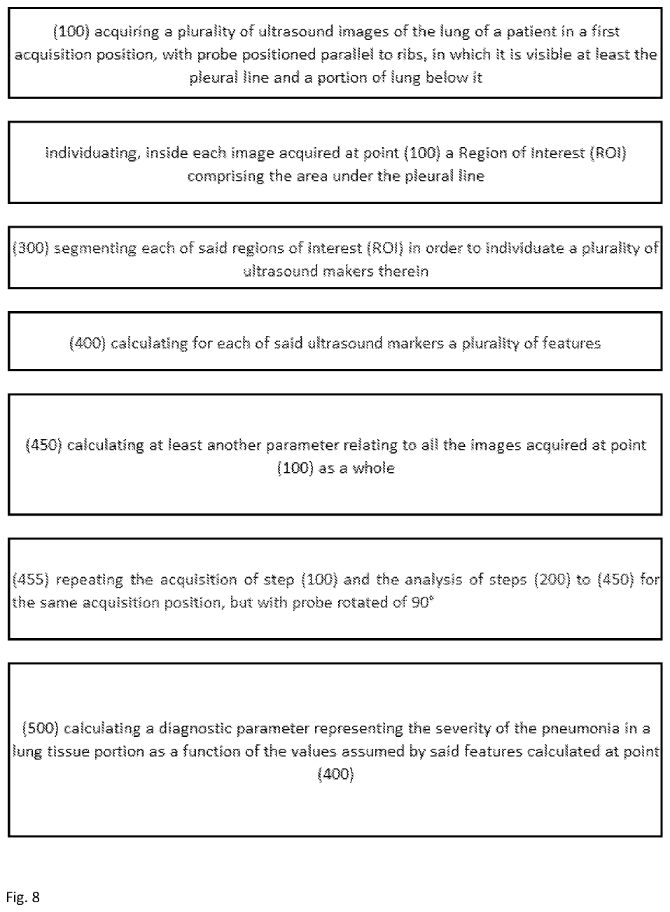

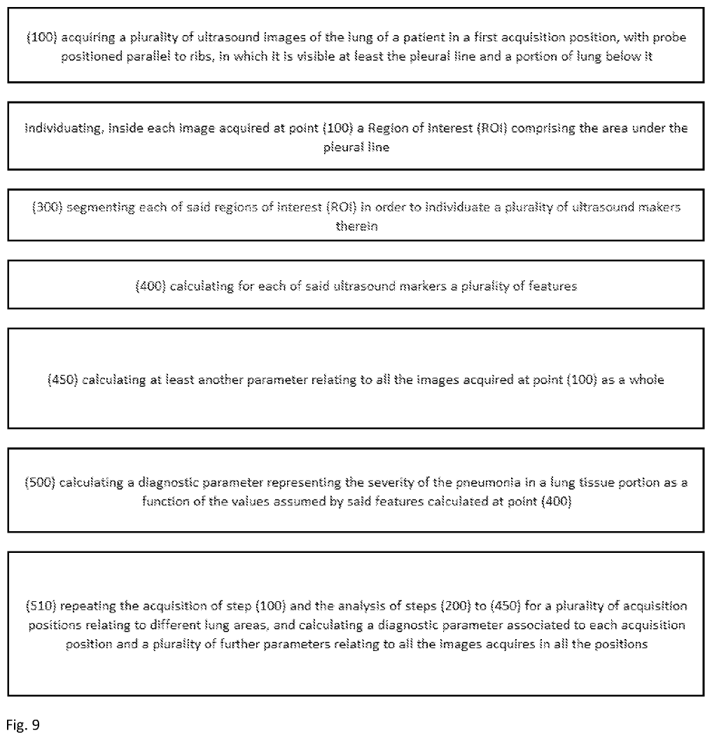

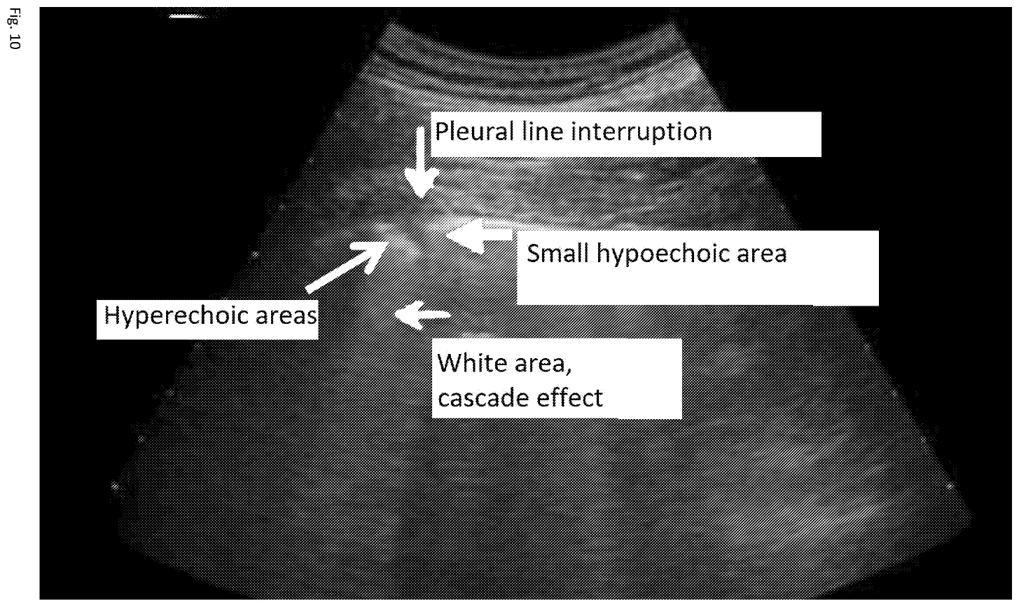

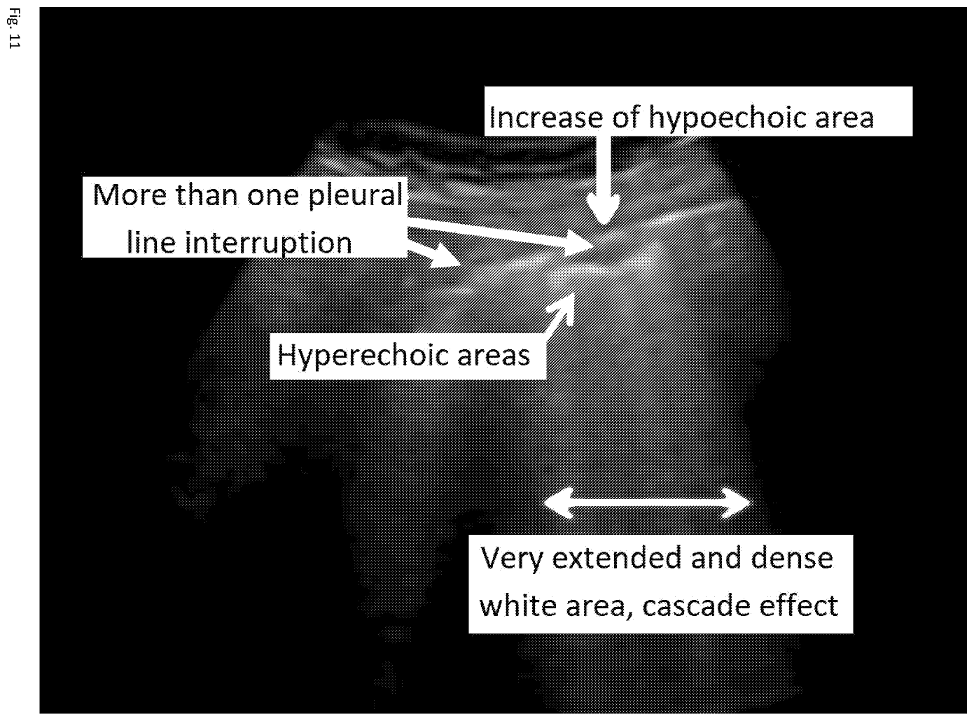

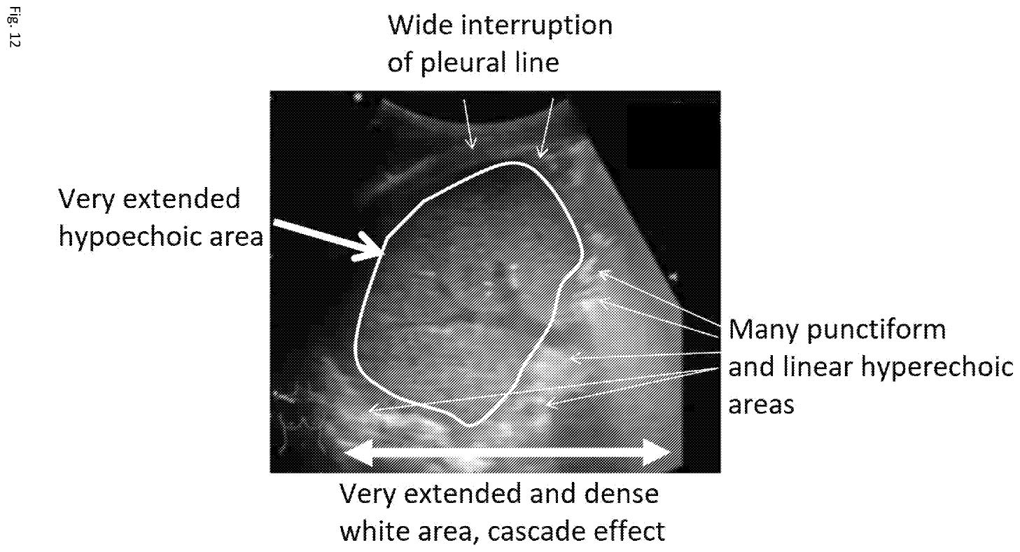

C4: consolidation areas (whose identification on a lung ultrasound image is shown in FIGS. 1 and 2). The consolidation areas can be identified by means of an automatic segmentation algorithm which, after the pleural line is identified, carries out the steps of:
(i) identifying the pleural line interruptions (by identifying possible interruptions of the bright profile relating to the pleural perimeter);
(ii) identifying the lung consolidations by searching hypoechoic areas in sub-pleural tissues adjacent to the interruptions detected at point (i);
(iii) identifying air bronchograms by searching possible punctiform and/or linear hyperechoic areas inside the consolidations detected at point (ii) or on their lower edge.

It is possible to define a set of features associated to the consolidations considered as a whole, which comprises one or more of the following ones: C₄₁: number of consolidations detected inside the ROI; C₄₂: average value of the averages of the shades of grey of the single consolidations; C₄₃: maximum value of the maximums of the shades of grey of the single consolidations; C₄₄: minimum value of the minimums of the shades of grey of the single consolidations; C₄₅: ratio between the average value of the averages of the shades of grey of the single consolidations and the average grey value of background.

It is also possible to define a set of features associated to each single consolidation, which comprises one or more of the following ones: C₄₆: average grey value of each consolidation; C47: maximum grey value of each consolidation; C₄₈: minimum grey value of each consolidation; C₄₉: ratio between the consolidation average grey value and background average grey value; C₄₁₀: minimum thickness of each consolidation; C₄₁₁: maximum thickness of each consolidation; C₄₁₂: average thickness of each consolidation; C₄₁₃: maximum depth of each consolidation; C₄₁₀: minimum depth of each consolidation; C₄₁₁: average depth of each consolidation; C₄₁₃: maximum width of each consolidation; C₄₁₄: minimum width of each consolidation; C₄₁₅: average width of each consolidation.

Preferably, but not limitingly, after segmenting the image in order to individuate the ultrasound marker relating to consolidations, the method provides also:
a. to analyze the frequency of the ultrasonic signal reflected by each consolidation, according to a technique commonly known as Color Doppler;
b. to define an ultrasonic signal frequency shift threshold, below which the consolidation is classified as “color-free signal”, and above which the consolidation is classified as “provided with color signal”,
c. to define the following feature assigned to each consolidation:

C₄₁₆: presence of color signal.

In an embodiment, a plurality of features among the ones relating to consolidations is used to determine a diagnostic parameter indicating that the pneumonia is caused by SARS-CoV-2 virus or by any other cause. This can be done by using a classification neural network, trained with the values of the features defined for consolidations and relating to ultrasound images of patients, whose diagnosis of pneumonia caused by SARS-CoV-2 virus or by any other cause is known.

### C5: Background

Once the areas relating to A-lines, B-lines and consolidations are rejected from the ultrasound image, the sub-pleural background is the remaining portion positioned below the pleural line. The features associated to the background comprise one or more of the following ones:

C₅₁: homogeneity of the image computed on the sole background; C₅₂: background average grey value; C₅₃: background maximum grey value; C₅₄: background minimum grey value; C₅₅: horizontal position of the center of gravity evaluated on background grey values; C₅₆: vertical position of the center of gravity evaluated on background grey values; C₅₇: ratio between the average grey value of the upper quadrant and the average grey value of the lower quadrant of the whole background.

At the end of step (400), each acquired image has been segmented in a plurality of areas containing respective ultrasound markers (pleural line, possible A-lines, possible B-lines, possible consolidations, background) and, in the following a set of quantitative parameters (features), representing each one of said ultrasound markers, has been assigned to each image.

Then, the method comprises the step of:
(450) calculating at least one further parameter relating to all the images acquired at point (100) as a whole.

Preferably, this at least one parameter comprises the lung tissue percentage interested by “white lung”.

In another embodiment, described in detail in the following, this at least one parameter comprises at least one parameter characteristic of the signal frequency spectrum relating to the consolidations.

It has to be specified that for “white lung” it is intended a wider hyperechoic area, where a plurality of B-lines is fused in a sole homogenous sub-pleural echoic area.

More specifically, the “white lung” is characterized by compact B-lines. Compact B-line refers to a type of ultrasound image where the existing dense B-line makes the acoustic shadows of the ribs disappear on the whole scanning area when the probe carries out the scan in a direction perpendicular to the ribs. “White lung” and compact B-line indicate acute alveolar-interstitial syndrome (AIS) and are caused by the presence of a great quantity of lung fluid (including the lung and alveolar interstitial fluid). The percentage of tissue interested by “white lung” can be preferably calculated in the following manner:
by calculating the average of the percentage of tissue interested by “white lung” in all the acquisitions with probe parallel to the ribs;
by calculating the average of the percentage of tissue interested by “white lung” in all the acquisitions with probe perpendicular to the ribs;
by calculating an average of the just obtained values.

In another embodiment, in order to obtain a more conservative indication, the percentage of tissue interested by “white lung” can be calculated by calculating the maximum, instead of the average, in each of the three above enlisted calculating steps. It has to be noted that the method according to the invention provides preferably a combination of longitudinal and transversal acquisitions and this leads to two specific advantages (in particular when using a convex type probe):
1) a greater portion of lung is visualized: the subdivision of each lung in 7 anatomical regions, a “double” (transversal mode+longitudinal mode) ultrasound acquisition being carried out in each one of them, guarantees the effective scan of the whole lung despite the presence of the rib cage, and simplifies the development of a dedicated and repeatable acquisition protocol, easily integrable in a software guiding the operator during the scan, so to make it objective;
2) it allows to identify immediately and unequivocally the areas characterized by “white lung” at the maximum stage (this represents the basis for calculating the aerated volume percentage, otherwise possible only by CT, which has direct implications on the patient prognosis): when, in the transversal visualization, the percentage of tissue interested by “white lung” is at least 70% and in the longitudinal visualization the percentage of “white lung” is such that the shadowing effect caused by ribs is eliminated, the whole anatomical region corresponding to this “double” acquisition is classified as “not well aerated” and this fact, combined with the results obtained in the other anatomical regions, will contribute to determine the percentage of “not well aerated lung”, which is a value directly linked to the prognosis about the course of the pneumonia and so to possible therapeutic choices, such as intensive care unit or sub-intensive care unit hospitalization or the administration of determined specific drugs.

Then the method comprises the step of:
(500) calculating a diagnostic parameter (Pneumonia Score) representing the severity of the pneumonia in a portion of lung tissue as a function of the features calculated at point (400) and of the further parameters calculated at step (450), characterized in that said diagnostic parameter is calculated with a two steps procedure:
a first step, in which to each ultrasound image acquired at point (100) a first value of the diagnostic parameter (Pneumonia Score) is assigned as a function of the number of A-lines, the number and configuration of B-lines and the pleural line continuity;
a second step, in which the average of the first values calculated for each image acquired at point (100) is corrected as a function of said further parameters calculated at point (450) and relating to all the images acquired in the first acquisition position.

Moreover, preferably, after step (450), the method comprises the step of:
(455) repeating the acquisition of step (100) and the analysis of steps (200) to (450) for the same acquisition position but with probe rotated of 90°, and is characterized in that the acquisitions are carried out with a convex type probe.

For example, if in the first acquisition position the probe is in transversal position (parallel to ribs), in the second acquisition position the probe is positioned in longitudinal position (orthogonal to ribs).

From an operational point of view, according to a first preferred embodiment:
it is acquired a plurality of images with longitudinal probe,
it is acquired a plurality of images with transversal probe,
it is calculated the diagnostic parameter in a scale 0 to 4 for each image,
it is calculated the trend of the calculated diagnostic parameters for the images acquired with transversal probe, the trend of the calculated diagnostic parameters for the images acquired with longitudinal probe and then it is calculated the average thereof,
it is increased the diagnostic parameter value if the average percentage of tissue interested by “white lung” relating all the acquired images is greater than a predetermined threshold, preferably equal to 70%.

Preferably, the parameter value is increased by one unit in a scale 0 to 4, in case of average percentage of tissue interested by “white lung” greater than the predetermined threshold.

Moreover, after step (500), the method comprises preferably the step of:
(510) repeating the acquisition of step (100) and the analysis of steps (200) to (500) for a plurality of acquisition positions relating to various lung areas, and calculating a diagnostic parameter associated to each acquisition position and a plurality of further parameters relating to all the images acquired in all the positions object of the analysis. These parameters are preferably:
the whole volume of all the consolidations detected in all the acquisition positions;
the percentage of lung tissue interested by “white lung”.
(550) Calculating a diagnostic parameter (Pneumonia Score) representing the severity of a pneumonia as a function of the features calculated at point (400) and further parameters calculated at step (510) by means of a two steps procedure:
a first step in which to the whole lung disease a first diagnostic parameter value is assigned as a function of the diagnostic parameter values associated to each acquisition position;
a second step in which said first diagnostic parameter value is corrected as a function of said further parameters calculated at point (510).

Preferably, with said correction the diagnostic parameter value is increased if the average percentage of tissue interested by “white lung” relating to all the acquired images is greater than a predetermined threshold, preferably equal to 70%. The diagnostic parameter value is then increased also if the whole volume of all the consolidations individuated in all the acquisition positions is greater than a predetermined threshold.

In the recent literature (Colombi et al., Radiology 2020, “Well-aerated Lung on Admitting Chest CT to Predict Adverse Outcome in COVID-19 Pneumonia”), it is known that a particularly important parameter in the prognosis of pneumonia is the aerated volume percentage. In fact, it was observed, for example, that patients having a “well-aerated” lung parenchyma percentage lower than 73% mostly went in intensive care unit and/or died. Vice versa, those whose “healthy” (so “well aerated”) lung tissue volume was greater than said threshold mostly had a positive course of the disease. Anyway, at the state of the art, the “well aerated” lung percentage can be measured only by CT.

The method according to present invention, even relying on the ultrasound technique, thanks to the just described two-step procedure, allows to distinguish between “well aerated” lung tissue portions and “not well aerated” lung tissue ones, since it uses synergically:
(i) information relating to each single image (markers individuated in each image);
(ii) information relating to other images acquired in the same position;
(iii) information relating to the same lung region of each image, but on orthogonal plane.

In particular, it is possible to classify as “not well aerated” each lung portion having a Pneumonia Score classification calculated at step (500) of severity greater than a predetermined threshold. Preferably, the threshold corresponds to a severity equal at least to 3, in a scale 0 to 4.

In this way, once the scan protocol based on the “double” acquisition of each one of the 14 lung regions (7 for each lung) is completed, it is possible to automatically calculate optimal estimate of the percentage of compromised lung volume, with resulting very important indications for healthcare personnel future decisions. As a way of example, we can say that for patients having the “not well aerated” region classification for at least 4 of the 14 lung regions, it will be assumed a particularly aggressive therapeutic approach and/or the prompt hospitalization in intensive care unit. According to a first embodiment, said diagnostic parameter is expressed by means of the classification of pneumonia in an advancement class chosen among five (or more) increasing severity classes, the first one of which corresponding to the absence of the disease.

Preferably, said plurality of acquisition positions of point (510) comprises one or more of the following ones, and preferably all the following positions:
1. right lung back portion scan, lower quadrant;
2. right lung back portion scan, middle quadrant;
3. right lung back portion scan, higher quadrant;
4. left lung back portion scan, lower quadrant;
5. left lung back portion scan, middle quadrant;
6. left lung back portion scan, higher quadrant;
7. right lung sub-axillary/lateral portion scan, lower quadrant;
8. right lung sub-axillary/lateral portion scan, higher quadrant;
9. left lung sub-axillary/lateral portion scan, lower quadrant;
10. left lung sub-axillary/lateral portion scan, higher quadrant;
11. right lung front portion scan, lower quadrant;
12. right lung front portion scan, higher quadrant;
13. left lung front portion scan, lower quadrant;
14. left lung front portion scan, higher quadrant.

Preferably, after step (550), the following steps are carried out:
(910) defining an asymmetry parameter of disease severity,
(920) defining the pneumonia as not caused by Sars-Cov-2 virus, if said asymmetry parameter is higher than a predetermined threshold.

In a first embodiment, said asymmetry parameter is calculated as the difference between the sum of Pneumonia Score values calculated for the acquisition positions relating to a lung and the sum of Pneumonia Score values calculated for the acquisition positions relating to the other lung.

In another embodiment, said asymmetry parameter is calculated as the ratio between the sum of Pneumonia Score values calculated for each acquisition position relating to a lung and the sum of Pneumonia Score values calculated for each acquisition positions relating to the other lung.

In another embodiment, the method provides further to calculate a statistical parameter indicating the probability that the pneumonia is caused by Sars-Cov-2 virus (Covid Index) as a function of the anamnestic information provided by the patient and of the value of the diagnostic parameters calculated for each acquisition position at step (500).

Such statistical parameter can be calculated:
by acquiring from the patient anamnestic information relating to the presence of other symptoms and possible conditions of exposure occurred recently;
by calculating a first partial Covid Index value as a function of the anamnestic information;
by summing to said partial Covid Index value, as a function of the anamnestic information, a second partial Covid Index value as a function of the analyses carried out at steps (100) to (550).

Preferably, the first partial Covid Index value is obtained by collecting from the patient information relating to the possible presence of a plurality of other symptoms, with a specific score being assigned to each of them, and relating to the possible occurrence of a plurality of conditions of exposure of the patient, with a specific score being assigned to each of them, and by summing up the scores assigned to each symptom present and to each condition of exposure really verified.

For example, symptoms and relative scores can be the following ones: fever: 3; sore throat: 4; presence of at least one symptom of respiratory disease (cough, shortness of breath, dyspnea): 7; total loss of sense of smell: 6; decreased sense of smell; 3: total loss of taste: 6; taste alteration: 3; diarrhea: 2.

The scores relating to various conditions of exposure can be the following ones: travels/staying in areas with infection incidence higher than a predetermined threshold within the previous 14 days: 5; in case of strict contact with a probable or confirmed case within the 14 days before the onset of symptoms: living with a confirmed COVID-19 case: 15; direct physical contact with COVID-19 case: 10; direct contact with COVID-19 case secretions (for example used handkerchiefs, cutlery, glasses, . . . ): 12; direct contact, for example face to face, with COVID-19 case: 9; staying in a closed room with COVID-19 case: 8; COVID-19 case samples handling for working reasons: 7; travel by the same transport means as a COVID-19 case: 7; direct contact with healthcare operators during home isolation: 5.

Said second partial Covid Index value is calculated preferably as a function of the Pneumonia Score values, in a scale 0 to 4, relating to each one of the previously enlisted 14 acquisition positions. With a Pneumonia Score other than 0 in scans relating to the lower quadrant, the second partial Covid Index value is calculated as the sum of the Pneumonia Scores relating to the back and lateral acquisition positions in the lower quadrant, with half the sum of the Pneumonia Scores relating to the other acquisition positions.

Otherwise, with Pneumonia Scores greater than 0 only in points other than the lower ones, said second partial Covid Index value is calculated as the sum of the Pneumonia Score values relating to the acquisition positions on the middle and higher quadrants.

### Method for Calculating the Diagnostic Parameter of Step (500)

While describing now the method for calculating the diagnostic parameter of step (500), it is to be specified what follows.

In a first embodiment, said first diagnostic parameter value is determined by using a classification neural network, configured to receive in input the values calculated for said features and to provide in output a vector containing the values of probability of belonging to each one of said classes and trained by using a set of parameters calculated in the just described manner, relating to lung ultrasound images of a plurality of patients whose disease advancement stage is known and for whom the class of belonging has been defined by skilled operators as a function of the ultrasound scan analysis, and/or as a function of the information derived from other diagnostic examinations, for example CT.

According to another embodiment, the first diagnostic parameter value is a numeric value representing the pneumonia severity. Conveniently, said numeric value can be expressed in a scale from 0 to 100, and is called Pneumonia Score for simplicity in the following.

The classification neural network provides in output a vector containing the probability of belonging to each class, wherein an interval of Pneumonia Score values is assigned to each class. Pneumonia Score is then calculated as a function of the probability of belonging to each class and of the values defining the lower and upper ends of each class.

For example, Pneumonia Score can be calculated as a function of the ends of the first and second class for probability of belonging, weighted as a function of the respective probabilities of belonging.

If, for example, the classes are defined as in the following table

| | Lower | Higher | |
|---|---|---|---|
| | Pneumonia | Pneumonia | |
| Class | Score | Score | Description |
| 0 | 0 | 20 | Disease absent |
| 1 | 20 | 40 | Disease in initial |
| | | | stage |
| 2 | 40 | 60 | Disease in |
| | | | intermediate stage |
| 3 | 60 | 80 | Disease in advanced |
| | | | stage |
| 4 | 80 | 100 | Disease in peak stage |

The following condition can occur, in which the classification neural network provides in output the vector indicated in the “Probability of belonging” column.

| | Lower | Higher | | |
|---|---|---|---|---|
| | Pneumonia | pneumonia | | Probability |
| Class | Score | Score | Description | of belonging |
| 0 | 0 | 20 | Disease absent | 0.13 |
| 1 | 20 | 40 | Disease in | 0.37 |
| | | | initial stage | |
| 2 | 40 | 60 | Disease in | 0.25 |
| | | | intermediate | |
| | | | stage | |
| 3 | 60 | 80 | Disease in | 0.17 |
| | | | advanced stage | |
| 4 | 80 | 100 | Disease in | 0.08 |
| | | | peak stage | |

The first two classes, in order of probability of belonging are class 1 (initial; Pneumonia Score between 20 and 40; probability 0.37) and class 2 (intermediate; Pneumonia Score between 40 and 60; probability 0.25). So, the Pneumonia Score can be calculated as the weighted average of the outer intervals of the two classes of variability, weighted with the respective probabilities of belonging.

$$Pneumonia Score = ( 0.25 \times 60 + 0.37 \times 20 ) / ( 0.25 + 0.37 ) = 36.1$$

In a first embodiment, the first Pneumonia Score value is calculated using a regression function associating a numeric value of the Pneumonia Score to a set of numeric values (“features”) calculated according to what just described:

Pneumonia Score=ƒ(C₁₁, . . . ,C₁ₙ, . . . ,C₂₁, . . . ,C₂ₙ,Cₙ₁, . . . Cₙₙ)

Conveniently, said regression function ƒ is estimated by using a set of parameters calculated in the just described manner, relating to lung ultrasound images of a plurality of patients whose disease advancement stage is known and for whom the class of belonging has been defined by skilled operators, as a function of the analysis of the ultrasound scans, and/or as a function of the information derived from other diagnostic examinations, as for example CT.

In a third embodiment, the first Pneumonia Score value is calculated by using a regression neural network, configured to receive in input the values of said features and to provide in output a Pneumonia Score parameter value and trained by using a set of parameters relating to lung ultrasound images of a plurality of patients whose disease advancement stage is known, and for whom the Pneumonia Score value has been defined by skilled operators as a function of the analysis of ultrasound scans, and/or as a function of information derived from other diagnostic examinations, for example CT. The regression neural network provides in output the diagnostic parameter value directly.

In a fourth embodiment, the Pneumonia Score is calculated by using a classification neural network trained by using a set of parameters relating to lung ultrasound images of patients whose disease advancement stage is known, and for whom the class of belonging has been defined by skilled operators, as a function of the analysis of ultrasound scans, and/or as a function of information derived from other diagnostic examinations, for example CT, and in the following by using a regression neural network, in whose input the output vector of the classification neural network is provided.

As yet said, in order to carry out the classification step, the classification neural network has to be suitably trained, according to techniques known per se at the state of the art, by using a set training of features relating to images, which:
(i) are acquired and processed by means of the same just described modes;
(ii) are relating to patients, whose exact classification in terms of disease staging is previously known.

The exact classification in terms of disease staging can be known in various manners. As a way of pure example, many patients and during the same day can be subjected to the same examination by means of other diagnostic techniques (radiography, high resolution CT) and on the basis of these examinations the exact disease staging can be defined, in this case using automatic or semi-automatic dedicated software; alternatively, on the lung ultrasound images obtained for same patients and during the same day, one or more skilled ultrasound operator can carry out a manual classification of the disease (i.e. based on the analysis of images in light of one's expertise, and on extraction the possible of quantitative parameters carried out manually on the images).

The effective network training can be evaluated, according to techniques known at the state of the art, by means of data relating to a “validation set” (relating to patients whose exact classification is known, but whose data are not used for the network training).

Yet, the structure of the neural network could be designed and optimized as well according to techniques known at the state of the art, and different configurations of neural networks can be used without departing from the scope of the invention.

### Method of Characterization Using Also the Radiofrequency Ultrasonic Signal

In the following, some further embodiments of the invention are described, which, in order to calculate a diagnostic parameter representing the progression stage of a pneumonia caused by SARS-CoV-2 virus, use also a plurality of parameters extracted by radiofrequency raw ultrasonic signal. It is to be specified that in all the embodiments described in the following (cfr. step 410) the radiofrequency raw ultrasonic signal is segmented in the time domain in order to extract only the portion relating to the ROI, i.e. the portion relating to the area under the pleural line. The thus extracted signal contains all the information relating to the area under the pleural line, also those normally lost in the following processing needed to obtain the ultrasound image: this is another characteristic of the method according to the present invention distinguishing it from all the diagnostic methods known at the state of the art based instead on the ultrasound image analysis. In another embodiment, after step (400) and before step (500), the method according to the invention comprises the following steps:
(410) extracting from each raw ultrasonic signal received by each one of said CMUT or piezoelectric transducers of step (100) the portion corresponding to the relative segment of ultrasound image contained inside the marker relating to the consolidations individuated at point (300).

It is to be repeated that the segmentation of the raw ultrasonic signal is carried out in the time domain and the segmented signal is the raw one, i.e. received by the ultrasonic probe and not yet object of the processing the ultrasound image is obtained with.

This step allows to obtain a first important result: all and only the information relating to the characteristics of the tissue present inside the consolidations are contained in the thus segmented signal, since the raw signal has not been processed yet, with the result of losing information, and at the same time by means of the segmentation in the time domain all the information relating to tissues not object of consolidations has been eliminated.

At the end of the segmentation process, it will be then obtained, for each consolidation individuated inside each acquisition, a matrix of P×N dimensions, where P is the number of points of RF raw signal corresponding to each consolidation individuated at point (300) and N is the number of ultrasonic signals present in each consolidation width.

Preferably, but not limitingly, after step (410) and before step (420), the method comprises the step of:
(415) filtering each signal extracted at point (410) with a band-pass filter.

Preferably, the passing band is between 1 and 18 MHz, but different extension frequency bands can be used to adapt better the procedure to different probe characteristics. Downstream of the filtering then, for each consolidation individuated inside each acquisition, it will be obtained a matrix of P×N dimensions, the same dimensions as the matrix obtained at the end of the segmentation process. Hence, the method comprises the step of:
(420) carrying out an analysis in the frequency domain of each raw ultrasonic signal extracted at point (410) by extracting a set of parameters characteristic of the signal in the frequency domain.

Said set of parameters characteristic of the signal in the frequency domain defined at point (420) is calculated after calculating for each raw signal extracted at point (410) the FFT (Fast Fourier Transform) of the signal, thus obtaining N frequency spectra.

Preferably, but not limitingly, said second set of parameters characteristic of the signal in the frequency domain is calculated after:
(i) calculating the module in dB of the absolute value of each spectrum associated to each consolidation, according to the formula:

Spectrum dB=20*log 10(abs(spectrum))

thus obtaining still N spectra;
(ii) calculating the average spectrum (by average of all the values relating to the same frequency) of the spectra obtained at point (i), thus obtaining an average spectrum associated to each consolidation.

Preferably, said spectrum is calculated in a frequency range between 1 and 5 MHz; in another embodiment, said spectrum is calculated in a frequency range between 6 and 12 MHz; in another embodiment, said spectrum is calculated in a frequency range between 10 and 18 MHz.

Then, it is carried out the calculation of the average spectrum relating to the consolidations, obtained as the average of all the average spectra associated to each consolidation.

On the average spectrum thus obtained, a set of parameters characteristic of the average spectrum relating to the consolidations is then calculated, which comprises one or more of the following parameters:
a) the maximum value (PEAK) of said average spectrum (dimensions 1×1);
b) the area of the spectrum, obtained by calculating the integral of the spectrum on the axis of frequencies in a determined frequency range (dimensions 1×1);
c) spectrum peak frequency, i.e. frequency for which the spectrum has its own maximum (dimensions 1×1),
d) −6 dB band start frequency (lowest frequency of the spectrum having value equal to −6 dB, after having normalized the average spectrum with peak at 0 dB) (dimensions 1×1);
e) −6 dB band end frequency at −6 dB (highest frequency of the spectrum having value equal to −6 dB, after having normalized the average spectrum with peak at 0 dB) (dimensions 1×1);
f) band width at −6 dB (difference expressed in Hz between −6 db band end frequency and −6 dB band start frequency) (dimensions 1×1);
g) spectrum slope (derived with respect to the frequency) calculated at a determined frequency (dimensions 1×1);
h) coefficients of a polynomial interpolating said average spectrum in a frequency range comprising said peak frequency (dimensions n×1).

In this embodiment, the method is characterized in that at step (500) said diagnostic parameter is calculated with a two steps procedure:
a first step in which to each ultrasound image acquired at point (100) a first Pneumonia Score value is assigned as a function of the number of A-lines, of the number and configuration of B-lines and of the continuity of pleural line,
a second step in which the average of the first values calculated for each image acquired at point (100) is corrected as a function of said further parameters calculated at point (450) and of said set of parameters characteristic of the average spectrum relating to the consolidations.

In this embodiment, the method according to the invention uses the so-called “lung paradox” at most, on whose basis the disease progression gradually “uncovers” the lung at the ultrasound analysis, meaning that while the disease advances, it opens “acoustic windows” which are absent in case of healthy lung, thus easing the study of the ill lung. In fact, as recently explained by Soldati et al. (Applied Sciences 2020, 10, 1570), the lung ultrasound can be assimilated to a standard ultrasound only in presence of consolidations, which can be briefly defined as air-free tissues. So, in this case, it becomes doubly advantageous to refer to spectra of raw signals relating to consolidations, since these ones not only characterize the identified ultrasound marker, but they contain also direct (and not indirect as in the case of all the other markers detectable in a lung ultrasound image) information about the effective properties of the corresponding tissue. With reference to the step of the spectrum extraction, it is to be specified that, preferably, said spectrum is filtered with a band-pass filter.

Preferably, the passing band is between 1 and 18 MHz, but different extensions of the frequency band can be used to adapt better the procedure to characteristics of various ultrasound probes.

It is to be specified that in a first preferred embodiment, the spectrum is extracted, relating to the segment contained in the ROI of each one of the signals received by each piezoelectric transducer included in the array of piezoelectric transducers of the ultrasound probe. In other words, each radiofrequency ultrasonic signal is segmented in the time domain to extract its portion relating to the Region of Interest (which, in a preferred embodiment, is all the portion of image positioned under the pleural line), and the frequency spectrum is calculated from the thus extracted portion.

1. A method for calculating a diagnostic parameter indicating the stage of a pneumonia, implementable by means of a computer program loaded on computing means associated to an ultrasound device, comprising the steps of:
(100) acquiring, by means of an ultrasound device provided with a probe comprising an array of CMUT or piezoelectric transducers each configured to emit an ultrasonic impulse directed to the tissues object of classification and to receive the raw ultrasonic signal reflected by the tissues of the patient in response to said ultrasonic impulse, a plurality of ultrasound images of the lung of a patient in a first acquisition position with probe positioned parallel to ribs, in which it is visible at least the pleural line and a portion of lung below it; (200) individuating, inside each image acquired at point (100), a Region of Interest (ROI) comprising the area under the pleural line;
(300) segmenting each of said regions of interest (ROI) in order to individuate a plurality of ultrasound makers (Ci, . . . , Cn) therein; (400) calculating for each of said ultrasound markers (Ci, . . . , Cn) a plurality of features (Cii, . . . , Cim, Cni, . . . , Cnm), wherein each feature is a descriptive parameter expressible by means of a numeric value;
(450) calculating at least another parameter relating to the images acquired at point (100) as a whole;
(500) calculating a diagnostic parameter representing the severity of the pneumonia in a portion of lung tissue as a function of the values assumed by said features calculated at point (400), characterized in that said ultrasound markers comprise pleural line, A-Lines, B-lines and consolidations, in that said at least further parameter comprises the percentage of pleural line interested by “white lung”, and in that said diagnostic parameter is calculated with a two steps procedure:
a first step, in which to each ultrasound image acquired at point (100) a first value of Pneumonia Score is assigned as a function of the number of A-lines, the number and configuration of B-lines and the pleural line continuity;
a second step, in which the average of the first values calculated for each image acquired at point (100) is corrected as a function of said at least further parameter calculated at point (450) and relating to all the images acquired in the first acquisition position.

2. The method according to claim 1, wherein, after step (450), comprising further the step of:
(455) repeating the acquisition of step (100) and the analysis of steps (200) to (450) for the same acquisition position but with probe rotated of 90°, and in that at step (500) said diagnostic parameter is calculated with a procedure providing:
to calculate the diagnostic parameter, in a scale from 0 to 4, for each image, to calculate the trend of the calculated diagnostic parameters for the images acquired by transversal probe positioning, the trend of the calculated diagnostic parameters for the images acquired by longitudinal probe positioning and so to calculate the average thereof, to increase by one unit the diagnostic parameter value if the average percentage of tissue interested by “white lung” relating to all the acquired images is greater than a predetermined threshold.

3. The method according to claim 1, wherein, after step (500), comprising further the steps of:
(510) repeating the acquisition of step (100) and the analysis of steps (200) to (450) for a plurality of acquisition positions relating to various lung areas, and calculating a diagnostic parameter associated to each acquisition position and a plurality of other parameters relating to all the images acquired in all the positions object of the analysis, said plurality of other parameters comprising the whole volume of all the consolidations detected in all the acquisition positions and the percentage of lung tissue interested by “white lung”,
(550) calculating a diagnostic parameter representing the severity of a pneumonia as a function of the features calculated at point (400) and of the other parameters calculated at point
(510) by means of a two steps procedure: a first step in which to the whole lung disease a first diagnostic parameter value is assigned as a function of the diagnostic parameter values associated to each acquisition position; a second step in which said first diagnostic parameter value is corrected as a function of said further parameters calculated at point (510).

4. The method according to claim 3, wherein said correction of first diagnostic parameter value is increased if the average percentage of tissue interested by “white lung” relating to all the acquired images is greater than a predetermined threshold, or if the whole volume of all the consolidations individuated in all the acquisition positions is greater than a predetermined threshold.

5. The method according to claim 1, wherein said diagnostic parameter is expressed by means of the classification of the pneumonia in an advancement class chosen between five or more increasing severity classes, the first one corresponding to the absence of disease.

6. The method according to claim 1, wherein said first diagnostic parameter value is determined by using a classification neural network, configured to receive in input the values calculated for said features and to provide in output a vector containing the values of probability of belonging to each one of said classes and trained by using a set of parameters calculated in the just described manner, relating to lung ultrasound images of a plurality of patients whose disease advancement stage is known and for whom the class of belonging has been defined by skilled operators as a function of the ultrasound scan analysis, and/or as a function of information derived from other diagnostic examinations, for example CT.

7. The method according to claim 6, wherein said classification neural network provides in output a vector containing the probability of belonging to each class, wherein to each class an interval of values of said diagnostic parameter is assigned, and in that said diagnostic parameter is calculated as a function of the probability of belonging to each class and of the values defining the lower and upper ends of each class.

8. The method according to claim 1, wherein said first diagnostic parameter value is calculated using a regression function associating to a set of numeric values (“features”), calculated according to what just described, a numeric value of the Pneumonia Score.

9. The method according to claims 1, wherein said first diagnostic parameter value is calculated by using a regression neural network, configured to receive in input the values of said features and to provide in output a Pneumonia Score parameter value and trained by using a set of parameters relating to lung ultrasound images of a plurality of patients whose disease advancement stage is known, and for whom the Pneumonia Score has been defined by skilled operators as a function of the ultrasound scan analysis, and/or as a function of information derived from other diagnostic examinations, for example CT.

10. The method according to claim 1, wherein said first diagnostic parameter value is calculated by using a classification neural network trained by using a set of parameters relating to lung ultrasound images of patients whose disease advancement stage is known, and for whom the class of belonging has been defined by skilled operators as a function of the ultrasound scan analysis, and/or as a function of information derived from other diagnostic examinations, for example CT, and subsequently a regression neural network, receiving as input the output vector of the classification neural network.

11. The method according to claim 1, after step (400) and before step (500), comprising further the following steps of:
(410) extracting from each raw ultrasonic signal received by each one of said CMUT or piezoelectric transducers of step (100), the portion corresponding to the relative segment of ultrasound image contained inside the marker relating to consolidations individuated at point (300),
(420) carrying out an analysis in the frequency domain of each raw ultrasonic signal extracted at point (410) by extracting a set of parameters characteristic of the signal in the frequency domain, and characterized in that at step (500) said diagnostic parameter is calculated with a two steps procedure:
a first step, in which to each ultrasound image acquired at point (100) a first Pneumonia Score value is assigned as a function of the number of A-lines, of the number and configuration of B-lines and of the pleural line continuity;
a second step, in which the average of the first values calculated for each image acquired at point (100) is corrected as a function of said at least further parameter calculated at point (450) and of said set of parameters characteristic of the average spectrum relating to consolidations.

12. The method according to claim 10, wherein, after step (410) and before step (420), comprising further the steps of:
(415) filtering each signal extracted at point (410) with a band-pass filter.

13. The method according to claim 3, wherein said plurality of acquisition positions of point (510) comprises one or more of the following ones, and preferably all the following positions:
1. right lung back portion scan, lower quadrant;
2. right lung back portion scan, middle quadrant;
3. right lung back portion scan, higher quadrant;
4. left lung back portion scan, lower quadrant;
5. left lung back portion scan, middle quadrant;
6. left lung back portion scan, higher quadrant;
7. right lung sub-axillary/lateral portion scan, lower quadrant;
8. right lung sub-axillary/lateral portion scan, higher quadrant;
9. left lung sub-axillary/lateral portion scan, lower quadrant;
10. left lung sub-axillary/lateral portion scan, higher quadrant;
11. right lung front portion scan, lower quadrant;
12. right lung front portion scan, higher quadrant;
13. left lung front portion scan, lower quadrant;
14. left lung front portion scan, higher quadrant.

14. The method according to claim 13, comprising further the calculation of a statistical parameter indicating the probability that the pneumonia is caused by Sars-Cov-2 virus (Covid Index), as a function of the anamnestic information provided by the patient and of the value of the diagnostic parameters calculated for each acquisition position at step (500).

15. The method according to claim 14, wherein said statistical parameter is calculated: by acquiring from the patient anamnestic information relating to the presence of symptoms and possible conditions of exposure occurred recently;
by calculating a first partial Covid Index value as a function of the anamnestic information;
by summing to said partial Covid Index value, as a function of the anamnestic information, a second partial Covid Index value as a function of the analysis carried out at steps (100) to (550).

16. The method according to claim 15, wherein said first partial Covid Index value is obtained by collecting from the patient information relating to the possible presence of a plurality of other symptoms, with a specific score being assigned to each of them, and to the possible occurrence of a plurality of patient conditions of exposure, with a specific score being assigned to each of them, and by summing up the scores assigned to each symptom present and to each condition of exposure really verified.

17. The method according to claim 16, wherein said second partial Covid Index value is calculated as a function of the Pneumonia Score values in a scale from 0 to 4, relating to each one of the 14 previously enlisted acquisition positions.

18. The method according to claim 17, wherein, in case of Pneumonia Score other than 0 in scans relating to the lower quadrant, said second partial Covid Index value is calculated as the sum of Pneumonia Scores relating to back and lateral acquisition positions in the lower quadrant, with half the sum of Pneumonia Score relating to other acquisition positions, said second partial Covid Index value being calculated instead as the sum of the Pneumonia Score value relating to the acquisition position on the middle and higher quadrants.

19. A ultrasound device comprising computing means on which computer programs are loaded, configured to carry out the method according to claim 1.
